(19) United States
(12) Patent Application Publication
(10) Pub. No.: US 2022/0112148 A1
(43) Pub. Date: Apr. 14, 2022

(54) PREPARATION METHOD FOR POLYOXYMETHYLENE DIMETHYL ETHER AND MIXTURE THEREOF

(71) Applicant: SHANDONG CHENXIN NEW ENERGY CO., LTD., Shandong (CN)

(72) Inventors: Yijin Cai, Shandong (CN); Xinglu Wang, Shandong (CN); Yichao Cai, Shandong (CN); Jizhen Zheng, Shandong (CN)

(73) Assignee: SHANDONG CHENXIN NEW ENERGY CO., LTD., Shandong (CN)

(21) Appl. No.: 17/560,796

(22) Filed: Dec. 23, 2021

Related U.S. Application Data
(63) Continuation of application No. PCT/CN2020/115358, filed on Sep. 15, 2020.

(30) Foreign Application Priority Data
Sep. 19, 2019 (CN) 201910884450.4
Sep. 27, 2019 (CN) 201910921323.7

(51) Int. Cl. C07C 41/56; C07C 41/58

(52) U.S. Cl. CPC C07C 41/56; C07C 45/38; C07C 41/58

(57) ABSTRACT

A preparation method for polyoxymethylene dimethyl ether (DMMn, generally, n=3-8 or 2-8) and mixture thereof may include one or more of the following: mixture of aqueous formaldehyde solution and polyalcohol is heated and dehydrated under vacuum conditions to yield flowing polyoxymethylene containing low moisture, which is heated and gasified to obtain relatively pure gaseous formaldehyde, which is mixed with methylal, previous batch of low-boiling-point substances applied, macromolecules, etc. in presence of catalyst. Or, the method mainly includes: aqueous formaldehyde solution, polyalcohol, and previous batch of macromolecular mixture applied are heated under vacuum conditions to remove moisture so as to obtain anhydrous flowable polyoxymethylene etherate, which is mixed with methylal, previous batch of low-boiling-point substances applied, macromolecules, etc. in presence of catalyst.

### CROSS-REFERENCE TO RELATED APPLICATION

The present disclosure is a U.S. continuation application of PCT Application No. PCT/CN2020/115358, filed on Sep. 15, 2020 and published as WO2021/052328 A1, which claims priority to the Chinese patent application filed with the Chinese Patent Office on Sep. 27, 2019 with the filing No. 201910921323.7, and entitled “Preparation Method for Polymethoxydimethyl Ether (DMMn)”, and the Chinese patent application filed with the Chinese Patent Office on Sep. 19, 2019 with the filing No. 201910884450.4, and entitled “Preparation Method for Polyoxymethylene Dimethyl Ether and Mixture thereof.” The contents of all applications listed in this paragraph are hereby incorporated herein by reference in their entireties.

### TECHNICAL FIELD

The present disclosure belongs to the technical field of energy and chemical industry, and particularly relates to a preparation method for Polymethoxydimethyl ether and a mixture thereof.

### BACKGROUND ART

Polymethoxydimethyl ethers, also called as polyoxymethylene dimethyl ethers, abbreviated as PODE or DMMn, are a type of low-molecular-weight acetal polymers with dimethoxymethane as a matrix and methyleneoxy as a main chain, and have a general formula represented by CH₃O(CH₂O)ₙCH₃. The polymethoxydimethyl ethers with the polymerization degree of 3-8 or 2-8, DMMn for short (n=2-8 or 3-8), are used for clean diesel oil blending components. The physical and chemical properties of the polymethoxydimethyl ethers are similar to those of diesel oil, then they can be blended into the diesel oil for use without the need of altering an oil supply system of a vehicle engine. The polymethoxydimethyl ethers have a cetane number of up to 76, and an oxygen content of 47%˜50%, and no sulfur or aromatic hydrocarbon. Adding 10%˜20% of the polymethoxydimethyl ether to the diesel oil can significantly reduce the cold filter plugging point of the diesel oil, may improve the combustion quality of the diesel oil in the engine, and improve the heat efficiency. Meanwhile, DMM3, DMM4, DMM5, and DMM3-8 are also solvents with extremely strong dissolving capacity. They may be applied in paints, coatings, inks, adhesives, cleaning agents, electrolyte solvents, and so on.

The polymethoxydimethyl ethers are generally prepared by reacting methanol or methylal with trioxymethylene or paraformaldehyde in the presence of an acidic catalyst, and basic equations of the reaction are as follows:

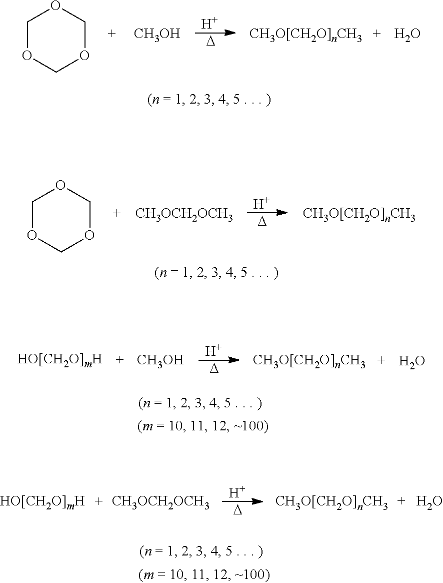

A process of synthesizing polymethoxydimethyl ether by catalysis of an acidic catalyst is an equilibrium reaction. The present of a small amount of water promotes the balance to move reversely, so that a large amount of formaldehyde, methanol, hemiacetal, and so on remain in the reaction liquid, and a content of a target product in a product mixture is lower, thus causing difficulty in purification and low-cost preparation of polymethoxydimethyl ethers. It is therefore conceivable to use formaldehyde with no water or low moisture content, and then it is conceivable to use trioxymethylene, paraformaldehyde, synthesized anhydrous or low-moisture gaseous formaldehyde, or to use relatively concentrated aqueous formaldehyde solution for synthesis and try to remove water of the system during the synthesis.

However, trioxymethylene is synthesized by concentrated formaldehyde under the condition of sulfuric acid catalysis. When there is water, a large amount of reflux is needed, gasification heat of water is great, energy consumption is high, and solvent extraction and dehydration are also needed, then the synthesis cost is caused higher. Moreover, the trioxymethylene, with a relatively high melting point (61° C.), is easy to sublimate, then pipeline is easy to clog, and dangerous accidents are easy to appear, and the like. For example, during storage and transportation, trioxymethylene will be polymerized to form a high molecular polymer once mixed with a strongly acidic substance, so that equipment is scrapped, the pipeline is destroyed, and dangerous accidents will occur.

Paraformaldehyde is in a solid form prepared by vacuum dehydration of an aqueous formaldehyde solution followed by polymerization, granulation or pulverization, drying, and other procedures. Although the production cost is reduced to some extent, when acting as a reactant, due to its insolubility and infusibility, the reaction activity is relatively low, and the charging is inconvenient from synthesis to application in the process of continuous production. Moreover, formaldehyde has stronger odor and higher toxicity, and is harmful to worker's health.

There are also reports on the preparation of polymethoxydimethyl ether (DMM3-8) through reaction between aqueous formaldehyde solution of a higher concentration and methanol or methylal in China. Although the process is theoretically smooth and convenient to operate, various technical problems exist in actual operation, for example, difficult re-dehydration of synthetic liquid, very low conversion rate, much residual formaldehyde, and difficult separation. The process is not mature, and can hardly realize industrialization.

It is reported that a DMMn synthetic liquid is prepared by catalytic reaction of gas formaldehyde and methylal in the presence of a catalyst, and DMM3-8 is obtained after treating and separating the DMMn synthetic liquid. However, in the preparation method of gaseous formaldehyde in this process, monohydric alcohol such as isobutanol is used as auxiliary agent, so that the atom utilization rate is low, the boiling point of the auxiliary agent is low, the auxiliary agent easily enters the synthetic liquid and finished product along with formaldehyde, moreover, the price is higher, which is not beneficial to industrialization. More importantly, such auxiliary agents, after forming hemiacetal, have increased water solubility, and can hardly layered with water, or special separation equipment is needed. In a subsequent distillation and dehydration process, an auxiliary additive reformed will be distilled out and mixed into the dilute formaldehyde solution due to the influence of the balance.

Formaldehyde gas is prepared by oxidizing methanol or methylal with air in an oxidation reactor; and the prepared formaldehyde gas is introduced into a cooler to be cooled to 20˜99° C., and then the formaldehyde gas is introduced into a gas-water separator to remove condensed water to obtain the formaldehyde gas. The fact proves that the formaldehyde gas formed by oxidizing the methanol contains about 30% of generated moisture, and when being reduced to 20˜99° C., it is easy to form formaldehyde hydrate to liquefy or generate polymerization reaction to liquefy or solidify, which is not beneficial to realizing industrialization.

Other conventional methods for preparing anhydrous gaseous formaldehyde include: 1. the gaseous formaldehyde is prepared by heating and depolymerizing paraformaldehyde, but the bound water contained in the paraformaldehyde itself is about 4%-12%, then the yield of DMMn synthesis and further recycling of intermediate products are greatly influenced. Not only is the preparation of the solid polyformaldehyde more complicated, but also the further charging and delivery are not beneficial to large-scale and continuous production, and there is great potential safety hazard. 2. Under the effect of an acidic catalyst, trioxymethylene is heated, decomposed, and gasified. In this way, trioxymethylene which is not decomposed in time will come out along with gasification of gaseous formaldehyde, then a delivery pipeline is easy to clog, moreover, the trioxymethylene has a higher cost, which is not beneficial to realizing industrialization.

### SUMMARY

The present disclosure provides a preparation method for polymethoxydimethyl ether (DMMn, preferably, n=3˜8), including the following steps:

mixing an aqueous formaldehyde solution and a polyalcohol according to a certain proportion, heating and dehydrating the resultant under vacuum condition to obtain flowing polyoxymethylene with a lower moisture content in one step,

heating and gasifying the flowing polyformaldehyde with a lower moisture content to obtain purer gaseous formaldehyde,

mixing the obtained gaseous formaldehyde, in the presence of a catalyst, with methylal, low-boiling-point substances and macromolecules recycled in the previous batch and so on, and then performing catalytic reaction at a certain temperature,

after reaching equilibrium of the reaction, filtering off the catalyst and purifying a synthetic liquid, followed by normal pressure distillation and reduced pressure distillation, respectively, to separate the low-boiling-point substances, the macromolecules, and a DMMn finished product, and

making the DMMn finished product then undergo reduced pressure rectification, wherein individual components of DMMn product may be obtained.

In the above, the polyalcohol remaining after the gasification of formaldehyde is returned, and used for concentration of formaldehyde of the next batch, so as to prepare the flowing polyoxymethylene with a lower moisture content.

In one or more embodiments, the polyalcohol includes, but is not limited to: ethylene glycol, 1,2-propylene glycol, 1,3-propylene glycol, diethylene glycol, glycerol, butanediol, neopentyl glycol, trimethylolpropane, or a mixture of two or more thereof.

In one or more embodiments, the aqueous formaldehyde solution includes: aqueous formaldehyde solution in a commercial form or gaseous formaldehyde containing water vapor, hydrogen, and methanol formed by catalytic dehydrogenation or oxidation of methanol.

In one or more embodiments, the aqueous formaldehyde solution has a formaldehyde content of 10%-85%, preferably 30%-70%.

In one or more embodiments, in the step of heating and dehydrating under vacuum condition, the mixture of formaldehyde and polyalcohol is dehydrated to a moisture content less than 15%, preferably less than 1.0%.

In one or more embodiments, in the step of heating and dehydrating under vacuum condition, a dehydration temperature of the mixture of formaldehyde and polyalcohol is 30° C.˜110° C., preferably 60° C.˜100° C.

In one or more embodiments, a temperature for heating and gasifying the flowing polyformaldehyde with a lower moisture content is 110° C.˜300° C., preferably 120° C.˜180° C.

In one or more embodiments, the catalyst is an acidic catalyst, and preferably the acidic catalyst includes: a liquid acidic catalyst, a solid acidic catalyst, or a gaseous acidic catalyst.

In one or more embodiments, the solid acidic catalyst includes: titanium silicalite molecular sieve, mordenite, sodium bisulfate, aluminum sulfate, ferric chloride, sulfonic acid resin, fluorosulfonic acid resin, silica gel particles adsorbed with sulfuric acid or phosphoric acid, or a mixture thereof.

In one or more embodiments, in the step of mixing an aqueous formaldehyde solution and a polyalcohol according to a certain proportion, a mass ratio of the formaldehyde to the polyalcohol is 1.0:0.02˜20.0, preferably 1:0.2˜2.0.

In one or more embodiments, a temperature of the catalytic reaction is 30° C.˜200° C., preferably 50° C.˜120° C.

The present disclosure provides a preparation method for polymethoxydimethyl ether (DMMn, preferably, n=2-8) and a mixture thereof, including the following steps:

mixing an aqueous formaldehyde solution, a polyalcohol, and macromolecules used in the previous batch according to a certain proportion, and then heating the resultant under vacuum condition to remove moisture, to obtain an anhydrous flowable polyoxymethylene etherate,

making the anhydrous polyoxymethylene etherate, in the presence of an acidic catalyst, with methylal, low-boiling-point substances and macromolecules recycled in the previous batch and so on, and then performing catalytic reaction at a certain temperature,

after reaching equilibrium of the reaction, filtering off the catalyst, followed by normal pressure distillation and reduced pressure distillation, respectively, to separate the low-boiling-point substances, the macromolecules, and a DMMn finished product,

making the DMMn finished product then undergo reduced pressure rectification, wherein individual components of DMMn product may be obtained; and

returning the low-boiling-point substances and macromolecules obtained by separation and using the same in the synthesis of the next batch.

In one or more embodiments, the polyalcohol includes: ethylene glycol, 1,2-propylene glycol, 1,3-propylene glycol, diethylene glycol, glycerol, butanediol, pentaerythritol, neopentyl glycol or trimethylolpropane.

In one or more embodiments, the aqueous formaldehyde solution includes: aqueous formaldehyde solution in a commercial form or gaseous formaldehyde containing water vapor formed by catalytic dehydrogenation or oxidation of methanol.

In one or more embodiments, the aqueous formaldehyde solution has a formaldehyde content of 5%-85%, preferably 30%-70%.

In one or more embodiments, the acidic catalyst includes: a liquid acidic catalyst, a solid acidic catalyst, or a gaseous acidic catalyst.

In one or more embodiments, the solid acidic catalyst includes: titanium silicalite molecular sieve, mordenite, sodium bisulfate, aluminum sulfate, ferric chloride, sulfonic acid resin, fluorosulfonic acid resin, silica gel particles adsorbed with sulfuric acid or phosphoric acid, or a mixture thereof.

In one or more embodiments, in the step of heating under vacuum condition to remove moisture, formaldehyde, polyalcohol, and macromolecules are dehydrated to a moisture weight less than 10 wt %, preferably less than 1.0 wt %.

In one or more embodiments, in the step of mixing an aqueous formaldehyde solution, a polyalcohol, and macromolecules used in the previous batch according to a certain proportion, a ratio of the mass of the formaldehyde and the mass of the polyalcohol and the macromolecule is 1.0:0.1˜10.0, preferably 1:0.2˜1.8.

In one or more embodiments, a temperature of the catalytic reaction is controlled at 30° C.˜180° C., preferably 50° C.˜80° C.

In one or more embodiments, the macromolecules are used for concentration of formaldehyde synthesized in the next batch or directly used for synthetic reaction.

### BRIEF DESCRIPTION OF DRAWINGS

In order to more clearly illustrate technical solutions of embodiments of the present disclosure, an accompanying drawing which needs to be used in the embodiments will be introduced briefly below, and it should be understood that the accompanying drawing below merely shows some embodiments of the present disclosure, and therefore should not be considered as limitation to the scope, a person ordinarily skilled in the art still could obtain other relevant drawings according to these accompanying drawings, without using creative efforts.

FIG. 1 shows a process flow of the present disclosure, i.e. an aqueous formaldehyde solution and a polyalcohol are mixed in a mixer, and then enter a vacuum dehydrator, flowing polyoxymethylene with a lower moisture content obtained after dehydration is then gasified in a gasifier to obtain purer gaseous formaldehyde, then, the obtained gaseous formaldehyde is mixed with methylal, low-boiling-point substances and macromolecules recycled in the previous batch and so on, the mixture enters a catalytic reactor, and then catalytic reaction is carried out in the presence of a catalyst at a certain temperature. After the reaction, a reaction liquid is purified, and then enters a separation system for separation, to separate the low-boiling-point substances, the macromolecules, and a DMMn finished product. The polyalcohol remaining after the formaldehyde gasification is returned to the mixer, and used for concentration of formaldehyde of the next batch, to prepare flowing polyoxymethylene with a lower moisture content. After a small amount of accumulated moisture is separated, the recovered low-boiling-point substances are returned to the system and mixed with the gaseous formaldehyde, to continue to participate in the catalytic reaction.

### DETAILED DESCRIPTION OF EMBODIMENTS

In order to make the objectives, technical solutions, and advantages of the embodiments of the present disclosure clearer, the technical solutions in the embodiments of the present disclosure will be described below clearly and completely. If no specific conditions are specified in the embodiments, they are carried out under normal conditions or conditions recommended by the manufacturer. If the manufacturers of reagents or apparatus used are not specified, they are conventional products commercially available.

Unless otherwise defined herein, scientific and technical terms used in the present disclosure should have meanings that are all commonly understood by those ordinarily skilled in the art. Exemplary methods and materials are described below, but methods and materials similar or equivalent to those described herein may also be used in the practice or testing of the present disclosure.

In order to overcome the practical problem existing in the preparation method for the known gas formaldehyde, the present disclosure provides a new synthetic technology route and process of gas formaldehyde and DMMn, and the whole process runs in a liquid or gaseous state. This method has readily available raw materials, is convenient for realizing continuity and automation, has higher total yield, higher purity of product, lower cost, little wastewater contamination, safety and environmental friendliness, and is suitable for industrial production.

In order to solve a series of current problems existing in synthesizing polymethoxydimethyl ether with paraformaldehyde, trioxymethylene or aqueous formaldehyde solution, the present disclosure provides a new synthetic technology route and process. This method has readily available raw materials, is convenient for realizing continuity and automation, has higher total yield, lower cost, little wastewater and little contamination, safety and environmental friendliness, and is suitable for industrial production.

In one or more embodiments, a preparation method for polymethoxydimethyl ether (DMMn, generally, n=3-8) is to mix an aqueous formaldehyde solution and a polyalcohol according to a certain proportion, and then heat and dehydrate the mixture under vacuum condition, to obtain flowing polyoxymethylene with a lower moisture content in such one step. The flowing polyoxymethylene with a lower moisture content is then heated and gasified to obtain purer gaseous formaldehyde, then, the obtained gaseous formaldehyde is mixed, in the presence of a catalyst, with methylal, low-boiling-point substances and macromolecules used in the previous batch and so on, to undergo catalytic reaction at a certain temperature. After reaching equilibrium of the reaction, the catalyst is filtered off and then a synthetic liquid is purified, followed by normal pressure distillation and reduced pressure distillation, respectively, to separate the low-boiling-point substances, the macromolecules, and a DMMn finished product, and the DMMn finished product is subjected to reduced pressure rectification to obtain individual components. The polyalcohol remaining after the formaldehyde gasification is returned, and used for concentration of formaldehyde of the next batch, to prepare flowing polyoxymethylene with a lower moisture content. After a small amount of accumulated moisture is separated, the recovered low-boiling-point substances are returned to the system, thus realizing circulating production of DMMn.

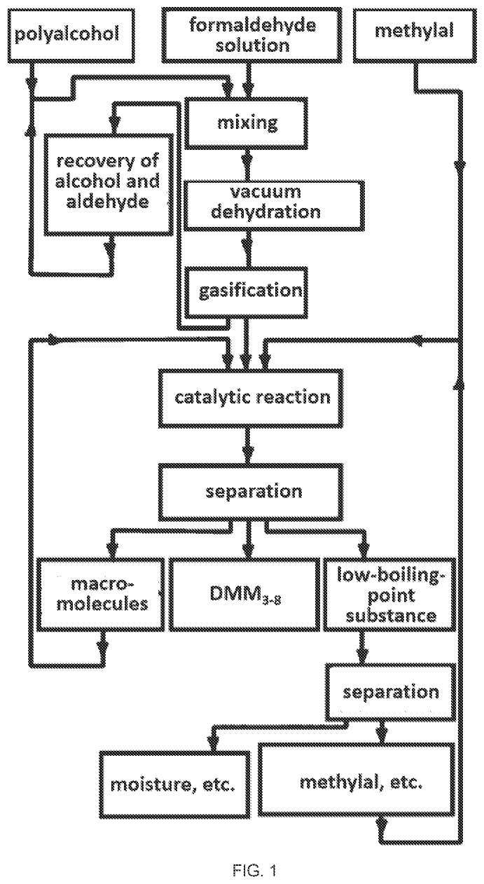

A basic flow is shown in the accompanying drawing (FIG. 1).

In the above, main reaction formulas for obtaining the flowing polyoxymethylene and gas formaldehyde are exemplified as follows:

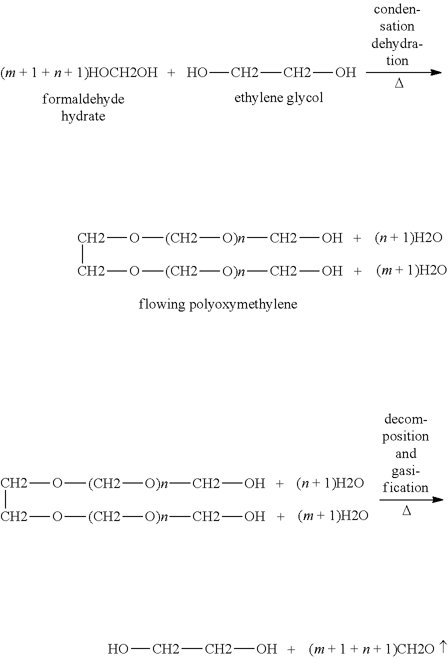

For the preparation method for polymethoxydimethyl ether (DMM3-8) in one or more embodiments of the present disclosure, the raw materials are readily available, the whole process is in a liquid state or a gas state, continuity and automation are conveniently realized, with higher yield, higher purity of product, lower cost, little wastewater contamination, safety and environmental friendliness. The present disclosure is suitable for industrial production.

A preparation method for polymethoxydimethyl ether and a mixture thereof in one or more embodiments of the present disclosure is to mix an aqueous formaldehyde solution, a polyalcohol, and macromolecules used in the previous batch according to a certain proportion, and then heat the mixture under vacuum condition to remove moisture, to obtain almost anhydrous, flowable polyoxymethylene etherate with low polymerization degree. The anhydrous etherate undergoes catalytic reaction with methylal, low-boiling-point substances and macromolecules used in the previous batch and so on at a certain temperature in the presence of a catalyst, after equilibrium of the reaction is reached, the catalyst is filtered off, followed by normal pressure distillation and reduced pressure distillation, respectively, to separate the low-boiling-point substances, the macromolecules, and a DMMn finished product. The DMMn finished product is subjected to reduced pressure rectification, then individual components may be obtained. The low-boiling-point substances and macromolecules obtained by separation are returned and used for synthesis of the next batch.

A basic flow of the reaction is as follows:

The preparation method for polymethoxydimethyl ether and a mixture thereof in one or more embodiments of the present disclosure have readily available raw materials, is simple and effective, and convenient to realize continuity and automation, has higher yield, lower cost, little wastewater contamination, safety and environmental friendliness, and is suitable for industrial production.

In one or more embodiments, the aqueous formaldehyde solution has a formaldehyde content of 10%-85%, for example, 15%, 20%, 25%, 30%, 35%, 40%, 45%, 50%, 55%, 60%, 65%, 70%, 75% or 80%.

In one or more embodiments, in the step of heating and dehydrating under vacuum condition, the mixture of formaldehyde and polyalcohol is dehydrated to a moisture content less than 15%, for example, less than 1.0%, for another example, 0.1-14, such as 13%, 12%, 11%, 10%, 9%, 8%, 7%, 6%, 5%, 4%, 3%, 2%, 1%, 0.8%, 0.6%, 0.4% or 0.2%.

In one or more embodiments, in the step of heating and dehydrating under vacuum condition, a dehydration temperature of the mixture of formaldehyde and polyalcohol is 30° C.˜110° C., for example, 35° C., 40° C., 45° C., 50° C., 55° C., 60° C., 65° C., 70° C., 75° C., 80° C., 85° C., 90° C., 95° C., 100° C. or 150° C.

In one or more embodiments, a temperature for heating and gasifying the flowing polyformaldehyde with a lower moisture content is 110° C.˜300° C., for example, 120° C., 130° C., 140° C., 150° C., 160° C., 170° C., 180° C., 190° C., 200° C., 210° C., 220° C., 230° C., 240° C., 250° C., 260° C., 270° C., 280° C. or 290° C.

In one or more embodiments, in the step of mixing an aqueous formaldehyde solution and a polyalcohol according to a certain proportion, a mass ratio of the formaldehyde to the polyalcohol is 1.0:0.02˜20.0, for example, 1:0.04, 1:0.06, 1:0.08, 1:1.10, 1:1.20, 1:1.40, 1:1.60, 1:1.80, 1:2.00, 1:3.00, 1:4.00, 1:5.00, 1:6.00, 1:7.00, 1:8.00, 1:9.00, 1:10.00, 1:11.00, 1:12.00, 1:13.00, 1:14.00, 1:15.00, 1:16.00, 1:17.00, 1:18.00 or 1:19.00.

In one or more embodiments, a temperature of the catalytic reaction is 30° C.˜200° C., for example, 40° C., 50° C., 60° C., 70° C., 80° C., 90° C., 100° C., 110° C., 120° C., 130° C., 140° C., 150° C., 160° C., 170° C., 180° C. or 190° C.

In one or more embodiments, the aqueous formaldehyde solution has a formaldehyde content of 5%-85%, for example, 10%, 15%, 20%, 25%, 30%, 35%, 40%, 45%, 50%, 55%, 60%, 65%, 70%, 75% or 80%.

In one or more embodiments, in the step of heating under vacuum condition to remove moisture, formaldehyde, polyalcohol, and macromolecules are dehydrated to a moisture weight less than 10%, for example, ₉%_{, 8}%_{,} 7%, ₆%_{, 5}%_{, 4}%_{, 3}%_{, 2}%_{, 1}%_{,} 0.8%, 0.6%, 0.4% or 0.2% (based on weight percentage).

In one or more embodiments, in the step of mixing an aqueous formaldehyde solution, a polyalcohol, and macromolecules used in the previous batch according to a certain proportion, a ratio of the mass of the formaldehyde and the mass of the polyalcohol and the macromolecule is 1.0:0.1˜10.0, preferably 1:0.2˜1.8.

It is embodied in the following aspects.

1. In one or more embodiments of the present disclosure, the aqueous formaldehyde solution (or gaseous formaldehyde synthesis gas) is used as a raw material of a formaldehyde source, so that the production is simplified, and the product cost is lower.

2. In one or more embodiments of the present disclosure, the polyalcohol is used as a carrier adjuvant to prepare a hemiacetal etherate, which has low cost, high efficiency, and easy operation, without contamination to subsequent materials.

3. One or more embodiments of the present disclosure solve the problem that polyformaldehyde, when being dehydrated to an anhydrous state, forms an insoluble and infusible low-activity solid state, which is hard to deliver.

4. The continuous process of one or more embodiments of the present disclosure is convenient to realize automated control.

5. The reaction process of one or more embodiments of the present disclosure has higher yield through appropriate control.

6. The production capacity of a single set of continuity device of one or more embodiments of the present disclosure can be made larger.

7. One or more embodiments of the present disclosure, using a solid acidic catalyst, has a good catalytic effect and is safe and environmentally friendly.

8. The formaldehyde of one or more embodiments of the present disclosure is still a liquid flowing easily after simple dehydration, facilitating realizing continuous production and automated control.

### Examples

Example 1: 400 g of aqueous formaldehyde solution (37%) and 60 g of ethylene glycol were mixed, and then vacuumized, and heated to remove moisture, to obtain 193.2 g of anhydrous flowing polyoxymethylene. The anhydrous flowing polyoxymethylene was then gradually heated to 160° C., the formaldehyde was gradually gasified to obtain gaseous formaldehyde, and then the dry anhydrous gaseous formaldehyde was introduced into a mixture of 10 g of catalyst and 280 g of methylal to undergo catalytic reaction at a certain temperature. After equilibrium of the reaction was reached, the catalyst was filtered off to obtain 356.7 g of reaction equilibrium liquid, and 91.7 g of ethylene glycol remained (24.8 g in total was lost). Then, 356.7 g of reaction equilibration liquid was subjected to normal pressure distillation and reduced pressure distillation, respectively, to separate 250.8 g of low-boiling-point substances, 15.5 g of macromolecules, and 72.4 g of DMMn finished product (18.0 g was lost). The DMMn finished product was subjected to reduced pressure rectification, then individual components may be obtained. The ethylene glycol remaining after the formaldehyde gasification was returned, and used for the concentration of formaldehyde of the next batch, to prepare the anhydrous flowing polyoxymethylene.

Example 2: 400 g of aqueous formaldehyde solution (37%) and 91 g of ethylene glycol recycled in the previous batch were mixed, and then vacuumized, and heated to remove moisture, to obtain 222.5 g of anhydrous flowing polyoxymethylene. The anhydrous flowing polyoxymethylene was then gradually heated to 160° C., the formaldehyde was gradually gasified to obtain gaseous formaldehyde, and then the dry anhydrous gaseous formaldehyde was introduced into a mixture of 10 g of catalyst and 280 g of methylal to undergo catalytic reaction at a certain temperature. After equilibrium of the reaction was reached, the catalyst was filtered off to obtain 384.7 g of reaction equilibrium liquid, and 93.5 g of ethylene glycol remained (24.3 g in total was lost). Then, 384.7 g of reaction equilibration liquid was subjected to normal pressure distillation and reduced pressure distillation, respectively, to separate 264.0 g of low-boiling-point substances, 22.9 g of macromolecules, and 77.3 g of DMMn finished product (20.5 g was lost). The DMMn finished product was subjected to reduced pressure rectification, then individual components may be obtained. The ethylene glycol remaining after the formaldehyde gasification was returned, and used for the concentration of formaldehyde of the next batch, to prepare the anhydrous flowing polyoxymethylene.

Example 3: 400 g of aqueous formaldehyde solution (37%) and 93.5 g of ethylene glycol recovered from the previous batch were mixed, and then vacuumized, and heated to remove moisture, to obtain 226.7 g of anhydrous flowing polyoxymethylene. The anhydrous flowing polyoxymethylene was then gradually heated to 160° C., the formaldehyde was gradually gasified to obtain gaseous formaldehyde, and then the dry anhydrous gaseous formaldehyde was introduced into a mixture of 10 g of catalyst, 264.0 g of recovered low-boiling-point substances, 22.9 g of macromolecules, and 196 g of additional methylal to undergo catalytic reaction at a certain temperature. After equilibrium of the reaction was reached, the catalyst was filtered off to obtain 593.6 g of reaction equilibrium liquid, and 90.0 g of ethylene glycol remained (26 g in total was lost). Then, 593.6 g of reaction equilibration liquid was subjected to normal pressure distillation and reduced pressure distillation, respectively, to separate 397.3 g of low-boiling-point substances, 35.5 g of macromolecules, and 128.8 g of DMMn finished product (32.0 g was lost). The DMMn finished product was subjected to reduced pressure rectification, then individual components may be obtained. The ethylene glycol remaining after the formaldehyde gasification was returned, and used for the concentration of formaldehyde of the next batch, to prepare the anhydrous flowing polyoxymethylene.

Example 4: 400 g of aqueous formaldehyde solution (37%) and 60 g of glycerol were mixed, and then vacuumized, and heated to remove moisture, to obtain 191.1 g of anhydrous flowing polyoxymethylene. The anhydrous flowing polyoxymethylene was then gradually heated to 160° C., the formaldehyde was gradually gasified to obtain gaseous formaldehyde, and then the dry anhydrous gaseous formaldehyde was introduced into a mixture of 10 g of catalyst and 280 g of methylal to undergo catalytic reaction at a certain temperature. After equilibrium of the reaction was reached, the catalyst was filtered off to obtain 360.7 g of reaction equilibrium liquid, and 92.0 g of glycerol remained (18.4 g in total was lost). Then, 360.7 g of reaction equilibration liquid was subjected to normal pressure distillation and reduced pressure distillation, respectively, to separate 246.8 g of low-boiling-point substances, 17.2 g of macromolecules, and 74.4 g of DMMn finished product (22.3 g was lost). The DMMn finished product was subjected to reduced pressure rectification, then individual components may be obtained. The glycerol remaining after the formaldehyde gasification was returned, and used for the concentration of formaldehyde of the next batch, to prepare the anhydrous flowing polyoxymethylene.

Example 5: 400 g of aqueous formaldehyde solution (37%) and 30 g of diethylene glycol, and 30 g of trimethylolpropane were mixed, and then vacuumized, and heated to remove moisture, to obtain 193.1 g of anhydrous flowing polyoxymethylene. The anhydrous flowing polyoxymethylene was then gradually heated to 160° C., the formaldehyde was gradually gasified to obtain gaseous formaldehyde, and then the dry anhydrous gaseous formaldehyde was introduced into a mixture of 10 g of catalyst and 280 g of methylal to undergo catalytic reaction at a certain temperature. After equilibrium of the reaction was reached, the catalyst was filtered off to obtain 357.7 g of reaction equilibrium liquid, and 94.5 g of a mixture of diethylene glycol and trimethylolpropane remained (20.9 g in total was lost). Then, 357.7 g of reaction equilibration liquid was subjected to normal pressure distillation and reduced pressure distillation, respectively, to separate 250.0 g of low-boiling-point substances, 20.5 g of macromolecules, and 73.4 g of DMMn finished product (13.8 g was lost). The DMMn finished product was subjected to reduced pressure rectification, then individual components may be obtained. The mixture of diethylene glycol and trimethylolpropane remaining after the formaldehyde gasification was returned, and used for the concentration of formaldehyde of the next batch, to prepare the anhydrous flowing polyoxymethylene.

### Comparative Example 1

400 g of aqueous formaldehyde solution (37%) was vacuumized under the same condition, and heated to remove moisture, to obtain 130.4 g of anhydrous non-flowable paraformaldehyde. The non-flowable paraformaldehyde was then gradually heated to 160° C., the formaldehyde was gradually gasified to obtain gaseous formaldehyde, and then the gaseous formaldehyde was introduced into a mixture of 10 g of catalyst and 280 g of methylal to undergo catalytic reaction at a certain temperature. After equilibrium of the reaction was reached, the catalyst was filtered off to obtain 387.8 g of reaction equilibrium liquid, and 0.6 g of solid residue remained (22 g in total was lost). Then, 387.8 g of reaction equilibration liquid was subjected to normal pressure distillation and reduced pressure distillation, respectively, to separate 295.9 g of low-boiling-point substances, 15.2 g of macromolecules, and 50.4 g of DMMn finished product (26.3 g was lost). The DMMn finished product was subjected to reduced pressure rectification, then individual components may be obtained. However, in this process, the formaldehyde was solid after polymerization, and was difficult to deliver and transfer. In a small scale intermittent experiment, the water content of paraformaldehyde obtained was still higher, up to 3.5%, which affected the equilibrium conversion rate of the synthetic liquid, and resulted in lower yield.

### Comparative Example 2

After 400 g of an aqueous formaldehyde solution (37%) and 60 g of isobutanol were mixed, they were miscible without delamination phenomenon, the mixture was vacuumized, and heated to remove moisture, to obtain 161.5 g of solid polyoxymethylene, with a formaldehyde content of 79.8, a moisture of 2.32%, and 31.2 g of isobutanol was lost. The solid polyoxymethylene is disadvantageous for operations such as delivery, and does not make sense to continue synthesis in the present experiment.

TABLE 1
Comparison between DMMn Finished Product Mass and
Lost Mass in Examples and Comparative Examples

| | Example | Example | Example | Example | Example | Comparative | Comparative |
|---|---|---|---|---|---|---|---|
| | 1 | 2 | 3 | 4 | 5 | Example 1 | Example 2 |
| DMMn | 72.4 | 77.3 | 128.8 | 74.4 | 73.4 | 50.4 | — |
| Finished | | | | | | | |
| Product | | | | | | | |
| Mass (g) | | | | | | | |
| Loss (g) | 18.0 | 20.5 | 32.0 | 22.3 | 13.8 | 26.3 | — |

It can be seen in Table 1 that the finished product mass in the examples is much higher than the finished product mass in Comparative Example 1 (as the solid polyoxymethylene in Comparative Example 2 is disadvantageous for operations such as delivery, this experiment cannot and does not make sense to continue). Moreover, the proportion of the mass loss to the finished product mass in the examples is also much lower than the result in Comparative Example 1.

### Example 6

800 g of 37% aqueous formaldehyde solution and 60 g of ethylene glycol were mixed, and water was removed under vacuum condition at 95° C. or less to obtain 326.4 g of anhydrous flowable polyoxymethylene etherate. The flowable polyoxymethylene etherate was transferred into a mixture having 10 g of acidic sulfonic acid resin and 560 g of methylal, to undergo catalytic reaction at a temperature of 50-80. After the reaction reached equilibrium, the catalyst was filtered off to obtain 866.4 g of reaction liquid. Firstly, 583.4 g of DMM1, DMM2, and so on were distilled off at normal pressure, and then 161 g of DMM3-8 was distilled off at −0.098 MP, and 97 g of macromolecules remained, which were used for synthesis of the next batch.

### Example 7

800 g of 37% aqueous formaldehyde solution and 120 g of ethylene glycol were mixed, and water was removed under vacuum condition at 95° C. or less to obtain 391.5 g of anhydrous flowable polyoxymethylene etherate. The flowable polyoxymethylene etherate was transferred into a mixture having 10 g of acidic sulfonic acid resin and 800 g of methylal, to undergo catalytic reaction at a temperature of 50-80. After the reaction reached equilibrium, the catalyst was filtered off to obtain 1152.5 g of reaction liquid. Firstly, 644.5 g of DMM1, DMM2, and so on were distilled off at normal pressure, and then 328 g of DMM3-8 was distilled off at −0.098 MP, and 158 g of macromolecules remained, which were used for synthesis of the next batch.

### Example 8

744.5 g of DMM1, DMM2, etc. obtained in the previous batch were separated specially under normal pressure to obtain 404.5 g of DMM1 (which may be used for synthesis of next batch); 25 g of methanol water (which may be used for methylal synthesis) was obtained; and 308.5 g of DMM2 was obtained (which may be used for synthesis of next batch).

### Example 9

800 g of 37% aqueous formaldehyde solution, 70 g of ethylene glycol, and 134 g of macromolecules of the previous batch were mixed, and water was removed under vacuum condition at 95° C. or less to obtain 443.8 g of anhydrous flowable polyoxymethylene etherate. The flowable polyoxymethylene etherate was transferred into a mixture having 10 g of acidic sulfonic acid resin, 920 g of methylal, and 279 g of recovered DMM2 and so on to undergo catalytic reaction at a temperature of 50-80. After the reaction reached equilibrium, the catalyst was filtered off to obtain 1621.8 g of reaction liquid. Firstly, 647.4 g of DMM1, 496.3 g of DMM2, and so on were distilled off at normal pressure, and then 323.5 g of DMM3-8 was distilled off at −0.098 MP, 119.6 g of macromolecules remained, which were used for synthesis of the next batch.

### Example 10

800 g of 37% aqueous formaldehyde solution and 60 g of glycerinum were mixed, and water was removed under vacuum condition at 95° C. or less to obtain 328.1 g of anhydrous flowable polyoxymethylene etherate. The flowable polyoxymethylene etherate was transferred into a mixture having 10 g of acidic sulfonic acid resin and 800 g of methylal to undergo catalytic reaction at a temperature of 50-80. After the reaction reached equilibrium, the catalyst was filtered off to obtain 1113 g of reaction liquid. Firstly, 742.5 g of DMM1, DMM2, and so on were distilled off at normal pressure, and then 236.5 g of DMM3-8 was distilled off at −0.098 MP, 112 g of macromolecules remained, which were used for synthesis of the next batch.

### Example 11

800 g of 37% aqueous formaldehyde solution and 60 g of diethylene glycol were mixed, and water was removed under vacuum condition at 95° C. or less to obtain 325.4 g of anhydrous flowable polyoxymethylene etherate. The flowable polyoxymethylene etherate was transferred into a mixture having 10 g of acidic sulfonic acid resin and 800 g of methylal to undergo catalytic reaction at a temperature of 50-80. After the reaction reached equilibrium, the catalyst was filtered off to obtain 1106.4 g of reaction liquid. Firstly, 744.9 g of DMM1, DMM2, and so on were distilled off at normal pressure, and then 235.5 g of DMM3-8 was distilled off at −0.098 MP, and 106 g of macromolecules remained, which were used for synthesis of the next batch.

### Example 12

800 g of 37% aqueous formaldehyde solution and 60 g of 1,2-propylene glycol were mixed, and water was removed under vacuum condition at 95° C. or less to obtain 327 g of anhydrous flowable polyoxymethylene etherate. The flowable polyoxymethylene etherate was transferred into a mixture having 10 g of acidic sulfonic acid resin and 800 g of methylal to undergo catalytic reaction at a temperature of 50-80. After the reaction reached equilibrium, the catalyst was filtered off to obtain 1110 g of reaction liquid. Firstly, 759 g of DMM1, DMM2, and so on were distilled off at normal pressure, and then 230 g of DMM3-8 was distilled off at −0.098 MP, and 102 g of macromolecules remained, which were used for synthesis of the next batch.

### Comparative Example 3

Water was removed from 800 g of 37% aqueous formaldehyde solution at 95° C. or less under vacuum condition to obtain 252 g of anhydrous non-flowable paraformaldehyde particles. The paraformaldehyde was transferred into a mixture having 10 g of acidic sulfonic acid resin and 800 g of methylal to undergo catalytic reaction at a temperature of 50-80. After 24 hours of reaction, there were still a large amount of paraformaldehyde particles, and it is impossible to separate the catalyst or to further obtain products.

The present disclosure solves the existing problem that the solid paraformaldehyde is hard to prepare and deliver and has low activity when the paraformaldehyde is taken as the raw material, the problem that the cost is high, equipment is severely corroded, and the pipeline has the risk of clogging when trimethylaldehyde is taken as the raw material, and a series of problems that the equilibrium conversion rate is low, the content of formaldehyde and hemiacetal is high, it is difficult to separate and obtain finished products, moisture accumulation makes circulating production impossible and so on when the aqueous formaldehyde solution is taken as a raw material. Under the circumstance that low-cost synthesis of DMMn cannot be achieved after a large number of researches on various synthetic routes and synthetic technologies of DMMn, the present disclosure is a technical result obtained by our company by developing a new way of thinking, through careful investigation and studious experimental verification, finally the present disclosure has readily available raw materials, simple and efficient process, lower cost, simple and smooth procedure, and higher synthetic conversion rate, and performs separation easily. The present disclosure has lower investment cost, a lower risk of production, and high profits.

Finally, it should be noted that the above-mentioned are merely preferred intermittent synthesis examples of the present disclosure, rather than being intended to limit the present disclosure. While the detailed description is made to the present disclosure with reference to the preceding examples, those skilled in the art still could modify the technical solutions recited in preceding examples, or make equivalent substitutions to some of the technical features therein, or only use the technology in the present disclosure to transform it into a continuous project. Any modifications, equivalent substitutions, improvements and the like made within the spirit and principle of the present disclosure are intended to be included within the scope of protection of the present disclosure.

### INDUSTRIAL APPLICABILITY

The present disclosure provides a preparation method for polymethoxydimethyl ether and a mixture thereof. In the present disclosure, an aqueous formaldehyde solution (or gaseous formaldehyde synthesis gas) is used as a raw material of formaldehyde source, which simplifies the production and reduces the product cost; using the polyalcohol as a carrier adjuvant to prepare a hemiacetal etherate has low cost, high efficiency, easy operation, and no contamination to subsequent materials; the problem that polyoxymethylene itself forms an insoluble and infusible low-activity solid state which is difficult to deliver when it is dehydrated to an anhydrous state is solved; the present disclosure is suitable for a continuous process, and convenient to realize automated control; the solid acidic catalyst may be used, with good catalytic effect, safety and environmental friendliness; formaldehyde is still a readily flowing liquid after simple dehydration, facilitating realizing continuous production and automated control.

We claim:

1. A method for preparing polymethoxydimethyl ether, comprising following steps:
mixing an aqueous formaldehyde solution and a polyalcohol according to a certain proportion, then heating and dehydrating a resultant under vacuum condition to obtain flowing polyoxymethylene with a lower moisture content in one step, heating and gasifying the flowing polyformaldehyde with a lower moisture content to obtain purer gaseous formaldehyde,
mixing obtained gaseous formaldehyde, in a presence of a catalyst, with methylal, low-boiling-point substances and macromolecules recycled in a previous batch, and then performing catalytic reaction at a certain temperature, filtering off the catalyst, after reaching equilibrium of reaction, and purifying a synthetic liquid, followed by normal pressure distillation and reduced pressure distillation, respectively, to separate the low-boiling-point substances, the macromolecules, and a DMMn finished product, and
making the DMMn finished product undergo reduced pressure rectification, so as to obtain individual components of DMMn product,
wherein the polyalcohol remaining after gasification of formaldehyde is returned and used for concentration of formaldehyde of a next batch, so as to prepare the flowing polyoxymethylene with a lower moisture content.

2. The method according to claim 1, wherein the polyalcohol comprises: ethylene glycol, 1,2-propylene glycol, 1,3-propylene glycol, diethylene glycol, glycerol, butanediol, neopentyl glycol, trimethylolpropane, or a mixture of two or more thereof.

3. The method according to claim 1, wherein the aqueous formaldehyde solution comprises: aqueous formaldehyde solution in a commercial form or gaseous formaldehyde containing water vapor, hydrogen, and methanol formed by catalytic dehydrogenation or oxidation of methanol.

4. The method according to claim 1, wherein the aqueous formaldehyde solution has a formaldehyde content of 10%-85%.

5. The method according to claim 1, wherein in a step of the heating and dehydrating a resultant under vacuum condition, a mixture of formaldehyde and the polyalcohol is dehydrated to a moisture content less than 15%.

6. The method according to claim 1, wherein in a step of the heating and dehydrating a resultant under vacuum condition, a dehydration temperature of a mixture of formaldehyde and the polyalcohol is 30° C.˜110° C.

7. The method according to claim 1, wherein a temperature for heating and gasifying the flowing polyformaldehyde with a lower moisture content is 110° C.˜300° C.

8. The method according to claim 1, wherein the catalyst is an acidic catalyst.

9. The method according to claim 8, wherein the acidic catalyst comprises: titanium silicalite molecular sieve, mordenite, sodium bisulfate, aluminum sulfate, ferric chloride, sulfonic acid resin, fluorosulfonic acid resin, silica gel particles adsorbed with sulfuric acid or phosphoric acid, or a mixture thereof.

10. The method according to claim 1, wherein in a step of the mixing an aqueous formaldehyde solution and a polyalcohol according to a certain proportion, a mass ratio of the formaldehyde to the polyalcohol is 1.0:0.02˜20.0.

11. The method according to claim 1, wherein a temperature of the catalytic reaction is 30° C.˜200° C.

12. A method for preparing polymethoxydimethyl ether and a mixture thereof, comprising following steps:
mixing an aqueous formaldehyde solution, a polyalcohol, and macromolecules used in a previous batch according to a certain proportion, and then heating a resultant under vacuum condition to remove moisture, to obtain an anhydrous flowable polyoxymethylene etherate,
making the anhydrous polyoxymethylene etherate, in a presence of an acidic catalyst, with methylal, low-boiling-point substances and macromolecules used in the previous batch, undergo catalytic reaction at a certain temperature,
filtering off the catalyst, after reaching equilibrium of reaction, followed by normal pressure distillation and reduced pressure distillation, respectively, to separate the low-boiling-point substances, the macromolecules, and a DMMn finished product,
making the DMMn finished product undergo reduced pressure rectification, so as to obtain individual components of DMMn product; and
returning the low-boiling-point substances and the macromolecules obtained by separation and using the same in synthesis of a next batch.

13. The method according to claim 12, wherein the polyalcohol comprises: ethylene glycol, 1,2-propylene glycol, 1,3-propylene glycol, diethylene glycol, glycerol, butanediol, pentaerythritol, neopentyl glycol or trimethylolpropane.

14. The method according to claim 12, wherein the aqueous formaldehyde solution comprises: aqueous formaldehyde solution in a commercial form or gaseous formaldehyde containing water vapor formed by catalytic dehydrogenation or oxidation of methanol.

15. The method according to claim 12, wherein the aqueous formaldehyde solution has a formaldehyde content of 5%-85%.

16. The method according to claim 12, wherein the acidic catalyst comprises: a liquid acidic catalyst, a solid acidic catalyst, or a gaseous acidic catalyst.

17. The method according to claim 16, wherein the solid acidic catalyst comprises:
titanium silicalite molecular sieve, mordenite, sodium bisulfate, aluminum sulfate, ferric chloride, sulfonic acid resin, fluorosulfonic acid resin, silica gel particles adsorbed with sulfuric acid or phosphoric acid, or a mixture thereof.

18. The method according to claim 12, wherein in a step of the heating a resultant under vacuum condition to remove moisture, formaldehyde, the polyalcohol, and the macromolecules are dehydrated to a moisture weight less than 10 wt %.

19. The method according to claim 12, wherein in a step of the mixing an aqueous formaldehyde solution, a polyalcohol, and macromolecules used in a previous batch according to a certain proportion, a ratio of a mass of formaldehyde and a mass of the polyalcohol and the macromolecules is 1.0:0.1˜10.0.

20. The method according to claim 12, wherein a temperature of the catalytic reaction is controlled at 30° C.˜180° C.
